(19) United States
(12) Patent Application Publication
(10) Pub. No.: US 2025/0090121 A1
(43) Pub. Date: Mar. 20, 2025

(54) JAW PROP DEVICE FOR MEDICAL IMAGING

(71) Applicant: CRANIO MANDIBULAR REHAB, INC., Denver, CO (US)

(72) Inventors: Robert Walter CHRISTENSEN, III, Denver, CO (US); Briana June BURRIS, Boston, MA (US); Joseph P. McCAIN, Boston, MA (US)

(21) Appl. No.: 18/887,954

(22) Filed: Sep. 17, 2024

Related U.S. Application Data
(60) Provisional application No. 63/539,305, filed on Sep. 19, 2023.

(51) Int. Cl. A61B 6/51

(52) U.S. Cl. CPC A61B 6/512

(57) ABSTRACT

A device may include a main body. A device may include a plurality of props, wherein each prop in the plurality of props includes a first biteplate and a second biteplate, the first biteplate and the second biteplate of each prop being in an inverted configuration with respect to each other, each of the first biteplate and the second biteplate of each prop includes a posterior dental stop rising from each of the first biteplate and the second biteplate, and further includes a spacer between the first biteplate and the second biteplate, wherein the spacer has a predetermined length. A device may include indicia on the main body indicating a dimension of at least one of the props, wherein each prop of the plurality of props extends from the main body in an arrangement wherein each prop is independent from the other props.

### CROSS-REFERENCE TO RELATED APPLICATIONS

This application claims priority to U.S. Provisional Patent Application No. 63/539,305 entitled “JAW PROP DEVICE FOR MEDICAL IMAGING” and filed on Sep. 19, 2024, which is hereby incorporated by reference.

### TECHNICAL FIELD

This invention generally relates to jaw prop or block devices, and more specifically to a jaw prop device that is used during medical imaging.

### BACKGROUND

There has long been a need for an apparatus that maintains the jaw in an open position. Common applications for such a device are during dental examinations and procedures. Additionally, an apparatus that holds the jaw open can be useful for stretching the tissues of the mouth, jaw, and joint as a means to reduce pain and increase opening of the mouth.

### SUMMARY

The present disclosure is about a bite prop for use during medical imaging to hold a user's mouth open to a repeatable and identifiable distance, in addition to other therapeutic benefits.

Certain aspects, advantages, and novel features have been described and may be summarized in whole or in part in the present disclosure. Not all of the present invention's advantages may be achieved in accordance with any one particular embodiment. Hence, the disclosed subject matter may be embodied or carried out in a manner that achieves or optimizes one advantage or group of advantages without achieving all advantages as may be taught or suggested.

In accordance with one embodiment, the present disclosure is for a jaw prop device that may be used to support a patient's jaw in an open position while medical imaging is being performed. Some embodiments of the device may include three or more props that are connected to a main body. Some embodiments of the device may also include a plurality of plates which contact the patient's teeth and hold the jaw open. Some embodiments of the device may include three or more bodies of clinically relevant heights, connected together. In some embodiments of the device, number indicators may be provided to allow the user to easily identify the magnitude of jaw opening that the corresponding prop provides.

Some aspects of the device may maintain the jaw of the patient in a predetermined open position. Furthermore, some aspects of the device allow the user to quickly determine what the magnitude of the patient's jaw opening is.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

These and other features, aspects and advantages of the present invention will become better understood with regard to the following description, appended claims, and accompanying figures where:

FIG. 1 shows a perspective view of a jaw prop device of a first embodiment.

FIG. 2 shows a side view of a jaw prop device of a first embodiment positioned in the mouth of a patient.

FIG. 3 shows a perspective view of a jaw prop device of a second embodiment.

FIG. 4 shows a perspective view of a jaw prop device of a third embodiment.

### DETAILED DESCRIPTION

Reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

As used in this disclosure, except where the context requires otherwise, the term “comprise” and variations of the term, such as “comprising”, “comprises” and “comprised” are not intended to exclude other additives, components, integers or steps. Additionally, in the following description, specific details are given to provide a thorough understanding of the embodiments. However, it will be understood by one of ordinary skill in the art that the embodiments may be practiced without these specific details. Well-known features, elements or techniques may not be shown in detail in order not to obscure the embodiments.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denote the same elements. As will be understood by those with skill in the art, the actual dimensions and proportions of any embodiment or element of an embodiment disclosed in this disclosure will be determined by its intended use.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

Conventional jaw prop or block devices include simple blocks, often rubber, inserted between the teeth in a posterior region of the mouth. Several disclosures describe static apparatuses that maintain the jaw open during dental procedures. These devices hold the jaw open at a single position, without a mechanism to adjust the magnitude of the opening. Typically, static jaw props have been proposed that are positioned in between the molars of the top teeth and the molars of the bottom teeth. These devices are difficult to insert and remove due to their position in the back of the mouth, are inconsistent across uses, and do not open the jaw to a specified distance between patients. Also, the size of the jaw opening is not specified or identifiable. Some dental props attempt to maintain the jaw in an open position via mechanical mechanisms, such as springs or screws. These devices are unnecessarily complex for imaging and consist of several different components. Since they require time to set up and adjust, they are inconvenient for many applications.

Existing, stepped devices can hold the jaw open at a large number of different distances, but the large number of possible opening distances provides a wide range of options for the imaging technician, causing inconsistency in the openings of the patient, unclear documentation of the opening, and increased risk for overstretching and injuring the user. These devices also extend or rotate into the mouth, possibly interfering with the imaging or irritating the user's mouth or tongue.

For diagnosis and surgical planning, doctors order imaging of the jaw and/or temporomandibular joint from radiologists or radiology technicians, such as X-ray or magnetic resonance imaging (MRI), which can cost hundreds to thousands of dollars for each session. These images are often conducted to diagnose disorders of the jaw, joint and the surrounding area. There are a limited number of solutions for propping the jaw open and no standardized protocol for specified opening distances.

Often, the radiologist does not know what opening to use and do not provide an image with the mouth open. Alternatively, radiologists often use makeshift solutions for taking an image with the patient's mouth open. One option is to have the patient hold their jaw manually open with their hand or jaw muscles while imaging is performed. Manual opening cannot hold a consistent static opening of the jaw for the extended duration of the scan, which leads to inaccurate imaging, motion artifacts in the images, unknown opening distance, and discomfort for the patient.

Examples of the makeshift options used by radiologist include a stack of tongue depressors, dental mouth props, bite blocks, an empty plastic syringe, or a pack of gauze between the teeth. The placement of these objects in the mouth is variable and are used inconsistently between patients, resulting in unknown or inaccurate distances of the mouth opening. The resulting images may then be diagnostically irrelevant, unusable by the ordering doctor, or may cause trauma or damage to the patient. Alternatively, these options require a secondary measurement of the opening, which may not be maintained through the imaging, and may not be consistent or diagnostically relevant. In such cases, the doctor will then require a second round of imaging requiring additional time and significant cost.

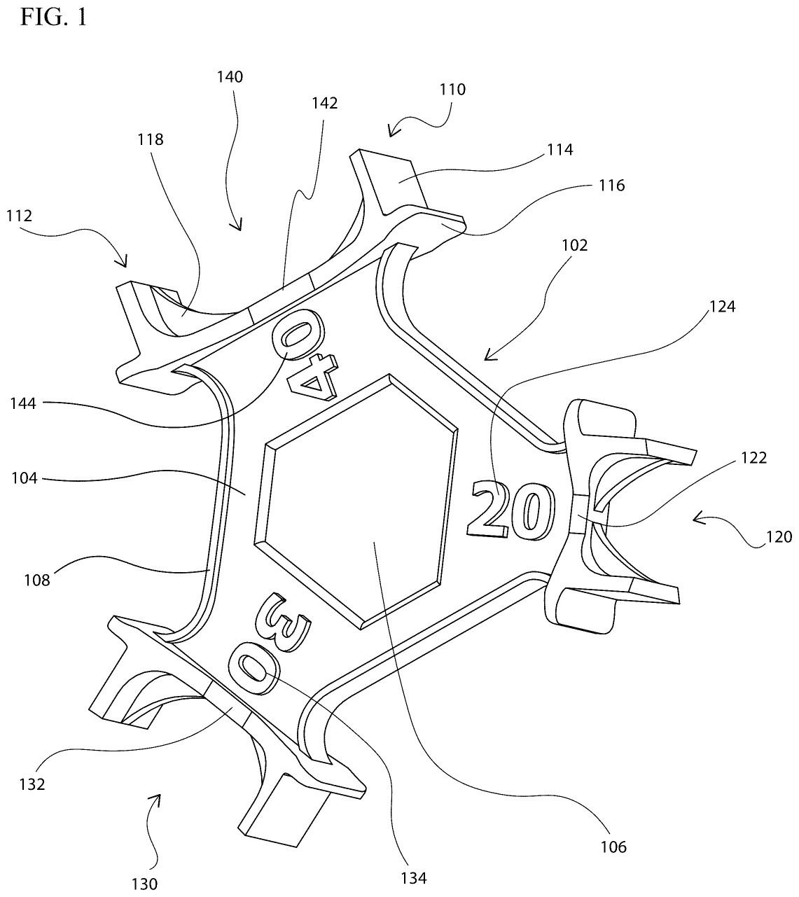

FIG. 1 shows a perspective view of a first embodiment of a jaw prop device. The jaw prop device is comprised of a main body 102 connected to a set of prop devices that includes the prop 120, the prop 130, and the prop 140. In the shown embodiment, prop 120 is configured to be a ‘small’ prop, prop 130 is configured to be a ‘medium’ prop, and prop 140 is configured to be a ‘large’ prop.

Each of the Small Prop 120, Medium Prop 130, and Large Prop 140 include a Mouthpiece portion 110 and an Inverted Mouthpiece portion 112, which are configured in inverse position to each other. Both the Mouthpiece 110 and Inverted Mouthpiece 112 consist of a Biteplate 114 for a row of teeth to rest on, a Retainer 116 to prevent the teeth from sliding over the Biteplate 114, and a Support 118 extending down from the Biteplate 114 for stability. In this embodiment, in each of the prop devices 120, 130, and 140, the Biteplate 114 is 12 mm wide and 3 mm thick. The Retainer 116 is 3 mm thick and rises 5 mm up from the Biteplate 114 with rounded corners. In this embodiment, the Support 118 is arcuate with a maximum height of 3 mm. In this embodiment, the biteplate 114 are rectangular and flat, but they could be formed in any shape, surface and texture that would engage a user's teeth or mouth while using the prop. The biteplates could further have an added surface material, pad or additional structure for cushioning or retention of the prop in the mouth.

The Mouthpiece portion 110 and an Inverted Mouthpiece portion 112 of each of the Small Prop 120, Medium Prop 130, and Large Prop 140 a separated by a spacer of a predetermined distance apart. The Small Prop 120 includes a Small Spacer 122 between the Mouthpiece 110 and Inverted Mouthpiece 112, the Small Spacer 122 being of a length so the distance between the Biteplate 114 of the Mouthpiece 110 and Inverted Mouthpiece 112 to create a prop size of 20 mm oral opening. The Medium Prop 130 includes a Medium spacer 132 being of a length to create a prop size of 30 mm oral opening. The Large Prop 140 includes a Large spacer 142 being of a length to create a prop size of 40 mm oral opening. The structure of the props could alternatively be understood as having a spine with the mouth piece and inverted mouthpiece extending out from the spine, such that where the spine portion between the mouthpieces would be the spacer and the spine rising beyond the mouthpieces would act as the retainer. In this configuration, the spine would of a selected length to equal the distance of the two spacers, plus the height of both mouthpieces, plus the spacer, to create a prop of predetermined distance.

The Main Body 102 may be a range of shapes and sizes. In this embodiment, the Main Body 102 consists of a Flat hexagonal plate 104 with a Void 106 in the center. In this embodiment, the Flat hexagonal plate 104 is 3 mm thick, and the Void 106 (also referred to herein as a removal structure) goes through the center of plate 104 in a predetermined size, suitable to fit a finger or hook inside it. The Small Prop 120, Medium Prop 130 and Large Prop 140 are connected to the Main Body 102 on every other side of the Flat hexagonal plate 104. A Rib 108 extends between the Small Prop 120, Medium Prop 130, and Large Prop 140 on the remaining alternate sides of the Flat hexagonal plate 104. In this embodiment the Rib 108 rises 1 mm from each side of the Flat hexagonal plate 104 for structural support. The rib height should be no more than 3 times the body thickness and the rib thickness should be 40-75% of the body thickness.

This embodiment further includes a Small Prop Indicator 124, a Medium Prop Indicator 134, and a Large Prop Indicator 144 one side of the Flat hexagonal plate 104. The Small Prop Indicator 124 is a raised “20” positioned adjacent to the Small Prop 120, the Medium Prop Indicator 134 is a raised “30” adjacent to the Medium Prop 130, and the Large Prop Indicator 144 is a raised “40” adjacent to the Large Prop 140. These indicia show the distance for each prop. In other embodiments, these indicia may be embossed, cut through, printed, or any other marking method on the Main Body 102; the indicia may be repeated or in an alternative orientation; the indicia may have radiopaque materials or qualities to show up on one or more imaging systems; the indicia may be of different sizes, fonts, or symbols, or incorporate high visibility materials or coatings for improved visibility.

The jaw prop device may be made of a variety of materials including but not limited to plastics and/or silicone. A range of manufacturing methods are feasible for producing the jaw prop device including injection molding, die cutting, thermoforming, compression molding, metal stamping, additive printing, etc.

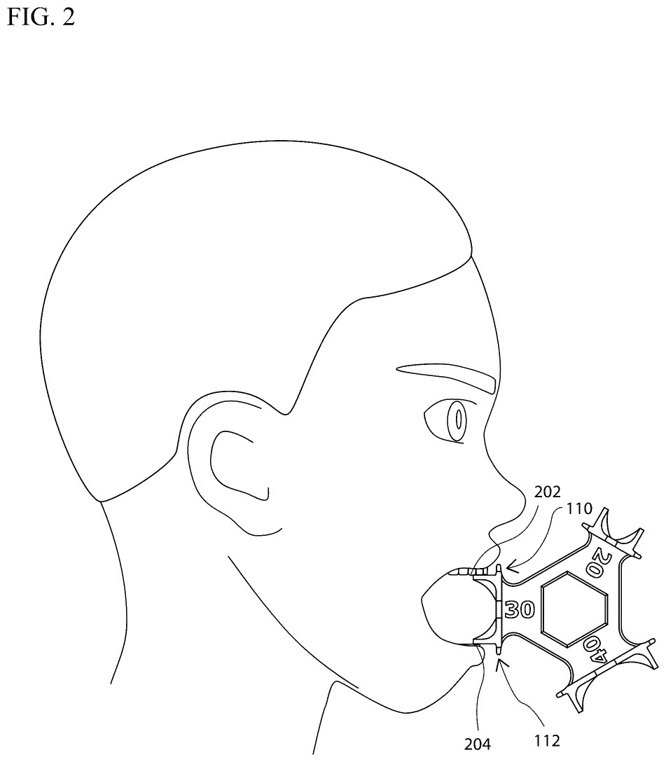

FIG. 2 shows a side view of the first embodiment of the jaw prop device positioned with the Medium Prop 130 in a user's mouth with one Mouthpiece 110 against the Upper Teeth 202 and the inverse Inverted Mouthpiece 112 resting on the Lower Teeth 204.

Before use of the jaw prop device, the user determines the appropriate open jaw position for the patient. Oftentimes, the user is a radiologist or a medical imaging technician. However, the user may be any individual capable of operating the device, including the patient themselves. The predetermined open jaw position depends on the limitations of the patient and preferences of the provider of care. The provider of care may be a radiologist, technician, or doctor, among others.

The predetermined distances of the Small Prop 120, Medium Prop 130 and Large Prop 140 are derived from clinically relevant distances of jaw opening for clear images of the temporomandibular joints (TMJ) in an open position, while minimizing the chance of damaging or injuring the patient. The TMJ are very small joints, located on the left and right sides of the skull. The jaw opens with first a rotation of the mandible in the two temporomandibular joints to about 20-25 mm. For openings beyond the jaw rotation, the mandible then slides down in the joints, in a motion called translation, to a maximum opening. The average jaw opening of an adult is approximately 50 millimeters between the upper and lower incisors. A functional jaw opening for adults has been shown to be above a 35 mm opening. Patients having less than a 35 mm oral opening are considered to have hypermobility of the jaw or trismus, with functional limitations on their use of the mouth that can impact their speaking, eating, drinking, oral hygiene, and access to dental and medical care.

However, a significant population of patients requiring images have damaged, degenerative, or painful joints. To reduce the risk of the user injuring the patient's joint by using forcing or overstretching the joint to insert the prop, the prop design should not provide too many options or include a maximum opening wider than necessary.

The jaw prop device was designed to simplify both the request of images of the jaw and TMJ by the clinician to the imaging tech, and the provision of those images by the tech, so the images provide a clear mouth openings for the clinician can see the joint with that mouth open, in a position called an “open bite”. To provide an image of the joint in a non-dysfunctional open position with the joint in translation, the prop requires a distance above 35 mm. For patients unable to open above the trismus range but capable of joint translation, a distance of below 35 mm but above 25 mm is needed. For patients with severely restricted jaw opening, the prop should show an opening with significant rotation of the joint, so that with the TMJs small size, the clinician can still see movement of the structures in the joint. From this anatomical and dysfunction measurements, we can see that clinically relevant distances can fall into broadly into 3 ranges: first, a lower range of 10-25 mm that allows a patient with limited opening to rotate the joint into an open position; second, a middle range from 20-40 mm, that shows a wider rotation of the joint without putting the joint into significant translation; and third an upper range of 35-55 mm allowing a user to show the joint in a non-dysfunctional full open position. For simplification, clarity and achieving the above opening ranges, distances of 20 mm, 30 mm, and 40 mm were selected for openings by this embodiment of the jaw prop device.

In this embodiment, the Small Prop 120 provides a conservative jaw opening of 20 millimeters for patients with very limited jaw mobility or strict limitations on their jaw opening. The Medium Prop 130 provides a mid-range jaw opening of 30 millimeters. The Large Prop 140 provides an opening of 40 millimeters, for situations where the patient has few limitations on their jaw opening and the user needs the jaw to be in a wide open position. The gradation of 10 millimeters between the Small Prop 120, Medium Prop 130, and Large Prop 140 provides a practical range of open jaw positions for the user to choose from, with unnecessary or inconsistent options.

To use the jaw prop device, the user opens their mouth as wide as possible with strain or stretching, and the appropriate prop inserted is between the patient's teeth to hold their mouth open. For example, if the patient's mouth can open to 37 mm, the Medium Prop 130 would be inserted into the patient's mouth. The Small Prop Indicator 124, Medium Prop Indicator 134, and Large Prop Indicator 144 allow the user or clinician to easily see and quickly identify the size of the Small Prop 120, Medium Prop 130 and Large Prop 140 used in the imaging for clear notation back to the requesting doctor.

The jaw prop device is placed in the mouth so that Mouthpiece 110 presses against the the Upper Teeth 202 and the Inverted Mouthpiece 112 down against the Lower Teeth 204. After insertion, the patient may relax and allow the jaw prop device to hold their jaw and mouth open. The Retainer 116 prevents the patient's teeth from sliding over and beyond the posterior edge of the biteplate 114, to prevent the prop from falling into the patient's mouth. The Support 118 provides extra support to each Biteplate 114 so they can withstand the force applied by the patient's jaw.

With the jaw prop device correctly placed, the user may proceed with the required medical imaging. Once the medical imaging is complete, the user opens their mouth so that the jaw prop device may be removed. If the device is stuck or in an emergency situation, a person can grasp the jaw prop device through the Void 106 and pull the jaw prop device out of the user's mouth. The jaw prop device can be manufactured as one continuous piece of material, to that it is economical to manufacture and feasible as a single-use device.

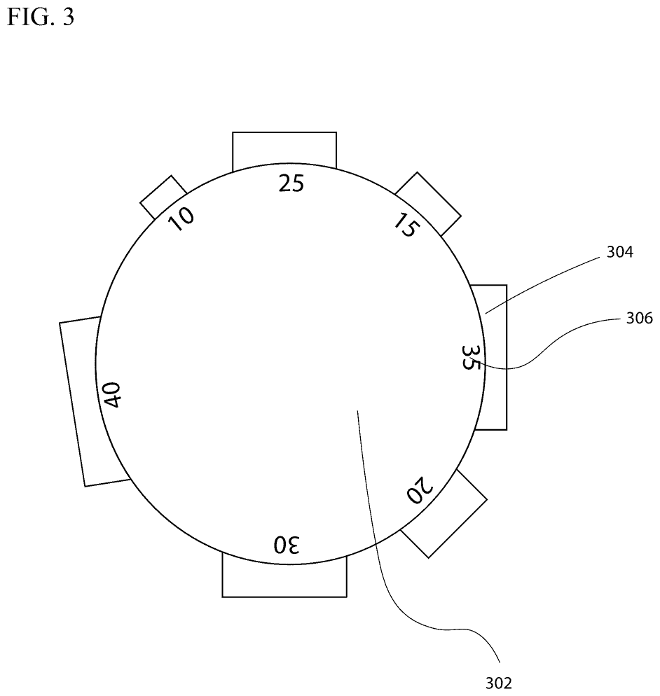

FIG. 3 shows a perspective view of an alternative embodiment of a jaw prop device. The jaw prop device of this embodiment comprises a Main Body 302 and a set of seven Props 304 in different widths with corresponding Indicia 306. In this embodiment, the Main Body 302 is a flat circular disc, and alternative embodiments could be any appropriate shape.

The props 304 of this embodiment are rectangular with predetermined distances between their sides, having lengths of 10 mm, 15 mm, 20 mm, 25 mm, 30 mm, 35 mm, and 40 mm. The Props 304 are arrayed around the Main Body 302, with their corresponding Indicia 306 located near each corresponding Prop 304 just inside the edge of the Main Body 302. The main body 302 must be of a size and shape such that the props 304 do not overlap or interfere with each other's usage. The Indicia 306 could further include other numerals, numbers, writing, etc., and they may be presented via embossing, printing, engraving, etc.

Alternative embodiments could include any predetermined number of independent and clinically relevant props 304 around a Main Body 302, with sufficient space and placement to allow the user to adequately insert one prop 304 into a user's mouth without interference from other props 304.

In this embodiment, the user opens their mouth and inserts the largest prop 304 that can fit comfortably between their teeth. The jaw prop device remains in the mouth until the imaging is complete and then is removed.

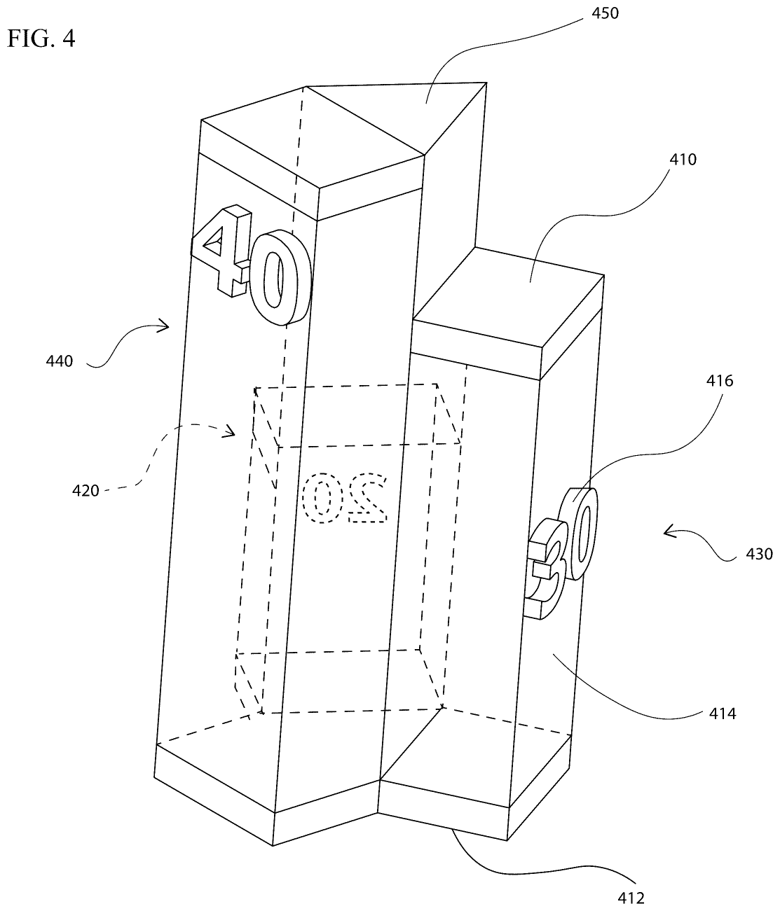

FIG. 4 shows a perspective view of an alternative embodiment of the jaw prop device. The jaw prop device of this embodiment comprises a 420 Small Prop, 430 Medium Prop, and Large Prop 440. In this embodiment, the 420 Small Prop, 430 Medium Prop, and Large Prop 440 are solid rectangular bodies connected at their long edges around a triangular Connecting Structure 450. Each of the 420 Small Prop, 430 Medium Prop, and Large Prop 440 includes an upper surface 410 and lower surface 412 separated by a body 414. Along the body 414 of each of the 420 Small Prop, Medium Prop 430 and Large Prop 440 are a prop marker 416, which are raised to show the distance of each prop. In this embodiment, each upper surface 410 and lower surface 412 are rectangular and extend out from the body 414. The upper surface 410 and lower surface 412 are of sufficient predetermined size to accommodate a user's front teeth. However, other shapes and configurations are possible.

In this embodiment, the distance between the upper surface 410 and lower surface 412, and thus jaw opening of each prop is 20 mm for the Small Prop 420 is, 30 mm for the Medium Prop 430, and 40 mm for the Large Prop 440. The components of the 420 Small Prop, Medium Prop 430 and Large Prop 440 are of sufficient thickness to maintain their shape under the pressure of the user's jaw.

In alternative embodiments, the 420 Small Prop, Medium Prop 430 and Large Prop 440 would be of different lengths, as short as 10 millimeters and as long as 45 millimeters. Further alternative embodiments of jaw prop device could have bite props made of bodies of other shapes, like C-shaped, cylindrical, or triangular bodies, or the device could include additional sized props with a different shaped or no connecting structure.

a. To operate this jaw prop device, the user opens their mouth and inserts the largest of the small prop 420, medium prop 430 or large prop 440 that fits comfortably between their teeth in their open mouth. The jaw prop device would remain in the mouth to hold it open during imaging, and is removed when complete.

Accordingly, the present disclosure provides a jaw prop device that meets the need for a simplified apparatus that assists users in positioning a patient's jaw at predetermined positions while medical imaging or other procedures are conducted. In particular, at least one of the embodiments of the jaw prop device provides a series of independent props that are an appropriate size and shape for placement on the front teeth of a patient, which allows for easy placement and removal. Additionally, the props include plates whose faces are a predetermined distance apart, with the predetermined distance of each prop marked by a numeral indicator. This configuration makes it easy for the user to quickly identify and notate the prop used for each patient.

At least one embodiment is made of a single continuous piece, which allows for economical manufacturing. As such, the jaw prop device may be provided to users at a reasonable price. Furthermore, the jaw prop device may be a single-use device, which alleviates the need for sterilization between patient uses.

In some aspects, the techniques described herein relate to a device, including: a main body; a plurality of props, wherein each prop in the plurality of props includes a first biteplate and a second biteplate, the first biteplate and the second biteplate of each prop being in an inverted configuration with respect to each other, each of the first biteplate and the second biteplate of each prop includes a posterior dental stop rising from each of the first biteplate and the second biteplate, and further includes a spacer between the first biteplate and the second biteplate, wherein the spacer has a predetermined length; indicia on the main body indicating a dimension of at least one of the props; wherein each prop of the plurality of props extends from the main body in an arrangement wherein each prop is independent from the other props.

In some aspects, the techniques described herein relate to a device, wherein the plurality of props includes only three props, a first distance between tops of the first biteplate of the first prop is in the range of 10 millimeters (mm) to 25 mm, a second distance between tops of the first biteplate of the second prop is in the range of 20 mm to 40 mm, and a third distance between tops of the first biteplate of the third prop is in the range of 35 mm to 55 mm.

In some aspects, the techniques described herein relate to a device, where the main body further includes a removal structure.

In some aspects, the techniques described herein relate to a device wherein the removal structure is a void through the main body, whereby the void is configured to receive a hook or a finger through the device.

In some aspects, the techniques described herein relate to a device, including: a set of jaw props of different sizes, each jaw prop including a body of predetermined height related to a jaw opening of no less than 10 mm and no more than 55 mm, with a perpendicular top and bottom shaped to sit on the teeth, wherein, each jaw prop in the set of jaw props is connected to another jaw prop in the set of jaw props in an arrangement such that each jaw prop can be inserted into a user's mouth independently, and whereby, each jaw prop in the set of jaw props is configured to support the jaw opening of a mouth to a predetermined distance between an upper and lower jaw of a patient in a predetermined open position.

In some aspects, the techniques described herein relate to a device, wherein the predetermined heights of the bodies of least three jaw props in the set of jaw props are in the ranges of 10 mm to 25 mm, 20 mm to 40 mm, and 35 mm to 55 mm.

In some aspects, the techniques described herein relate to a device, where the jaw props of the set of jaw props have bodies of rectangular shapes, all being connected along their long edges in a connecting structure.

In some aspects, the techniques described herein relate to a device, including: a flat main body; a first mouth prop extending out from an edge of the main body; a second mouth prop extending from the edge of the main body; a third mouth prop extending from the edge of the main body; wherein, the first mouth prop, the second mouth prop, and the third mouth prop each include a spine and two mouthpieces, the first and second mouthpieces being in inverse position with respect to each other to form inverted mouthpieces and extending out from the spine such that the mouthpieces create a mouth prop of a predetermined distance.

In some aspects, the techniques described herein relate to a device, wherein a distance between the mouthpieces of the first mouth prop is in the range of 10 mm to 25 mm, a distance between the mouthpieces of the second mouth prop is in the range of 20 mm to 40 mm, and a distance between the mouthpieces of the third prop is in the range of 30 mm to 55 mm.

In some aspects, the techniques described herein relate to a device, wherein the two inverted mouthpieces are located a predetermined distance from ends of the spine of each mouth prop, whereby the ends of the spine act as a posterior dental retainer preventing teeth of a first user from sliding too far over the mouthpiece.

In some aspects, the techniques described herein relate to a device, wherein the main body is a hexagon including six sequential edges, and the first mouth prop extends from the first edge of the six sequential edges, the second mouth prop extends from the third edge of the six sequential edges, and the third mouth prop extends from the fifth edge of the six sequential edges.

In some aspects, the techniques described herein relate to a device, wherein the main body includes a grip structure, whereby the grip structure allows a second user to grab and pull the device from the mouth of the first user.

In some aspects, the techniques described herein relate to a device, wherein said grip structure includes a void through the main body, wherein the void is of sufficient size to receive at least one finger, whereby a person could grip the device through the void to pull the device from within the mouth.

While the above description contains many specificities, these should not be construed as limitations on the scope of the invention, but as exemplifications of the presently preferred embodiments thereof. Many other ramifications and variations are possible within the teachings of the invention. For example, the main body may have any number of sides; the sides may be of a variety of shapes, such as curved, zig-zag, grooved, etc; the surfaces may be duplicated to include more than two; the surfaces may be different shapes; the number indicators may be a variety of sizes, formats, and orientations. The main body may include additional aspects that connect the plurality of sides and surfaces in alternative configurations. More so, the jaw prop device may be any range of colors and the jaw prop device may be made of any material.

Thus the scope of the invention should be determined by the appended claims and their legal equivalents, and not by the examples given.

The drawings and associated descriptions provided herein are intended to illustrate potential embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

1. A device, comprising:
a main body;
a plurality of props, wherein each prop in the plurality of props includes a first biteplate and a second biteplate, the first biteplate and the second biteplate of each prop being in an inverted configuration with respect to each other, each of the first biteplate and the second biteplate of each prop includes a posterior dental stop rising from each of the first biteplate and the second biteplate, and further includes a spacer between the first biteplate and the second biteplate, wherein the spacer has a predetermined length;
indicia on the main body indicating a dimension of at least one of the props;
wherein each prop of the plurality of props extends from the main body in an arrangement wherein each prop is independent from the other props.

2. The device of claim 1, wherein the plurality of props includes only three props, a first distance between tops of the first biteplate of the first prop is in the range of 10 millimeters (mm) to 25 mm, a second distance between tops of the first biteplate of the second prop is in the range of 20 mm to 40 mm, and a third distance between tops of the first biteplate of the third prop is in the range of 35 mm to 55 mm.

3. The device of claim 1, where the main body further includes a removal structure.

4. The device of claim 3 wherein the removal structure is a void through the main body, whereby the void is configured to receive a hook or a finger through the device.

5. A device, comprising:
a set of jaw props of different sizes, each jaw prop including a body of predetermined height related to a jaw opening of no less than 10 mm and no more than 55 mm, with a perpendicular top and bottom shaped to sit on the teeth,
wherein, each jaw prop in the set of jaw props is connected to another jaw prop in the set of jaw props in an arrangement such that each jaw prop can be inserted into a user's mouth independently, and
whereby, each jaw prop in the set of jaw props is configured to support the jaw opening of a mouth to a predetermined distance between an upper and lower jaw of a patient in a predetermined open position.

6. The device of claim 5, wherein the predetermined heights of the bodies of least three jaw props in the set of jaw props are in the ranges of 10 mm to 25 mm, 20 mm to 40 mm, and 35 mm to 55 mm.

7. The device of claim 5, where the jaw props of the set of jaw props have bodies of rectangular shapes, all being connected along their long edges in a connecting structure.

8. A device, comprising:
a flat main body;
a first mouth prop extending out from an edge of the main body;
a second mouth prop extending from the edge of the main body;
a third mouth prop extending from the edge of the main body;
wherein, the first mouth prop, the second mouth prop, and the third mouth prop each include a spine and two mouthpieces, the first and second mouthpieces being in inverse position with respect to each other to form inverted mouthpieces and extending out from the spine such that the mouthpieces create a mouth prop of a predetermined distance.

9. The device of claim 8, wherein a distance between the mouthpieces of the first mouth prop is in the range of 10 mm to 25 mm, a distance between the mouthpieces of the second mouth prop is in the range of 20 mm to 40 mm, and a distance between the mouthpieces of the third prop is in the range of 30 mm to 55 mm.

10. The device of claim 8, wherein the two inverted mouthpieces are located a predetermined distance from ends of the spine of each mouth prop, whereby the ends of the spine act as a posterior dental retainer preventing teeth of a first user from sliding too far over the mouthpiece.

11. The device of claim 8, wherein the main body is a hexagon including six sequential edges, and the first mouth prop extends from the first edge of the six sequential edges, the second mouth prop extends from the third edge of the six sequential edges, and the third mouth prop extends from the fifth edge of the six sequential edges.

12. The device of claim 8, wherein the main body includes a grip structure, whereby the grip structure allows a second user to grab and pull the device from the mouth of the first user.

13. The device of claim 12, wherein said grip structure includes a void through the main body, wherein the void is of sufficient size to receive at least one finger, whereby a person could grip the device through the void to pull the device from within the mouth.
